SEQUENCE LISTING (14 sequences)

SEQ ID NO: 1 - DNA, 1457 bp, Bacillus licheniformis
  misc_feature (62)..(63): Unkown nucleotide
ctcctgccaagctgaagcggtctattcatactttcgaactgaacatttttctaaaacagttnntaataaccaaaaaattttaaattggccctccaaaaaaataggcctaccatataattcattttttttctataataaattaacagaataattggaatagattatattatccttctatttaaattattctgaataaagaggaggagagtgagtaatgatgaggaaaaagagtttttggcttgggatgctgacggccttcatgctcgtgttcacgatggcattcagcgattccgcttctgctgctcaaccggcgaaaaatgttgaaaaggattatattgtcggatttaagtcaggagtgaaaaccgcatctgtcaaaaaggacgtcatcaaagagagcggcggaaaagtggacaagcagtttagaatcatcaacgcagcaaaagcgaagctagacaaagaagcgcttaaggaagtcaaaaatgatccggatgtcgcttatgtggaagaggatcatgtggcccatgccttggcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataacaccgacggcaacggacacggcacacatgttgccggtacagtagctgcgcttgacaatacaacgggtgtattaggcgttgcgccaagcgtatccttgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaagaggggttgtcgttgtagctgcagcagggaacagcggatcttcaggaaacacgaatacaattggctatcctgcgaaatacgattctgtcatcgctgttggtgcggtagactctaacagcaacagagcttcattttccagtgtgggagcagagcttgaagtcatggctcctggcgcaggcgtatacagcacttacccaacgaacacttatgcaacattgaacggaacgtcaatggtttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaataacatattctaacaaatagcatatagaaaaagctagtgtttttagcactagctttttcttcattctgatgaaggttgtccaatattttgaatccgttccatgatc

SEQ ID NO: 2 - protein, 379 aa, Bacillus licheniformis
  misc_feature (62)..(63): Unkown nucleotide
MMRKKSFWLGMLTAFMLVFTMAFSDSASAAQPAKNVEKDYIVGFKSGVKTASVKKDVIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMVSPHVAGAAALILSKHPNLSASQVRNRLSSTATYLGSSFYYGKGLINVEAAAQ

SEQ ID NO: 3 - DNA, 36 bp, Artificial sequence
  Synthetic oligonucleotide
cgaacggggtaccctcctgccaagctgaagcggtct

SEQ ID NO: 4 - DNA, 26 bp, Artificial sequence
  Synthetic oligonucleotide
cgcggatcctgagcggcagcttcgac

SEQ ID NO: 5 - DNA, 26 bp, Artificial sequence
  Synthetic oligonucleotide
cgcggatccctccaaggactcgaagg

SEQ ID NO: 6 - DNA, 39 bp, Artificial sequence
  Synthetic oligonucleotide
acgcacgcggatcccggcatcaccggcacctggtacaac

SEQ ID NO: 7 - DNA, 40 bp, Artificial sequence
  Synthetic oligonucleotide
acatacatgcatgcgagctctactgctgaacggcgtcgag

SEQ ID NO: 8 - DNA, 39 bp, Artificial sequence
  Synthetic oligonucleotide
cacatacatgcatgcttacggcttacacttggtgaaggt

SEQ ID NO: 9 - DNA, 27 bp, Artificial sequence
  Synthetic oligonucleotide
catgccatggcgcaaaccgttccttac

SEQ ID NO: 10 - DNA, 27 bp, Artificial sequence
  Synthetic oligonucleotide
ccgctcgagttgagcggcagcttcgac

SEQ ID NO: 11 - DNA, 36 bp, Artificial sequence
  Synthetic oligonucleotide
cgcacgcggatcctcgacattgatcagacctttccc

SEQ ID NO: 12 - DNA, 31 bp, Artificial sequence
  Synthetic oligonucleotide
tcagcatgccatggctgctcaaccggcgaaa

SEQ ID NO: 13 - DNA, 28 bp, Artificial sequence
  Synthetic oligonucleotide
tcagcatgccatggtgaggaaaaagagt

SEQ ID NO: 14 - DNA, 33 bp, Artificial sequence
  Synthetic oligonucleotide
ggacatcgagctcgacggcttcaccttggtgaa

(19) United States
(12) Patent Application Publication
(10) Pub. No.: US 2003/0108991 A1
(43) Pub. Date: Jun. 12, 2003

(54) Immobilization of keratinase for proteolysis and keratinolysis

(76) Inventors: Jason C.H. Shih, Cary, NC (US); Jeng-Jie Wang, Raleigh, NC (US); Harold E. Swaisgood, Raleigh, NC (US)

Correspondence Address: MYERS BIGEL SIBLEY & SAJOVEC, PO BOX 37428, RALEIGH, NC, 27627, US

(21) Appl. No.: 10/202,339

(22) Filed: Jul. 24, 2002

Related U.S. Application Data
(60) Provisional application No. 60/307,494, filed on Jul. 24, 2001.

(51) Int. Cl. C12Q 1/68; C07H 21/04; C12N 9/50; C12N 1/21; C12N 15/74

(52) U.S. Cl. 435/69.1; 435/219; 435/252.31; 435/320.1; 536/23.2; 435/6

(57) ABSTRACT

A recombinant nucleic acid encoding a fusion protein wherein the recombinant nucleic acid comprises a nucleic acid encoding a keratinase fused to a nucleic acid encoding a first member of a specific binding pair is described. An immobilized keratinase comprising a fusion protein and a solid support is also described. A method of digesting substrates such as keratin (e.g., feather) or protein (e.g., casein) is also described herein.

### RELATED APPLICATIONS

This Application claims the benefit of and priority from U.S. Provisional Application No. 60/307,494, filed Jul. 24, 2001, the disclosure of which is incorporated by reference herein in its entirety.

### FIELD OF THE INVENTION

The present invention relates to fusion proteins of a protein or keratinase and a binding partner, the immobilization thereof on solid supports, proteolysis and keratinolysis, and collection of the degradation products thereof.

### BACKGROUND OF THE INVENTION

Keratinaceous materials are often included in animal feeds as an inexpensive source of dietary protein. Keratins such as feathers, horns, hooves, and hair are readily available as agricultural by-products. A problem with feeding animals such materials, however, is that they are difficult to digest (Adler-Nissen, Enzymatic Hydrolysis of Food Proteins (1986). Elsevier Applied Science Publishers, New York, N.Y. p. 100). Hence, the amount of amino acids taken up into animals from such materials is relatively small compared to the total amount of amino acids present in such materials.

Feathers, in particular, are produced in large quantities by the poultry industry. These feathers provide an inexpensive source of raw material for a variety of potential uses. Among other things, there has been considerable interest in developing methods of degrading feathers so they can be used as an inexpensive source of amino acids and digestible protein in animal feed. To date, processes for converting feathers into animal feed include both steam and hydrolysis processes, and combined steam hydrolysis and enzymatic processes. See, e.g., Papadopoulos (1986) Animal Feed Science and Technology 16:151; Papadopoulos (1985) Poultry Science 64:1729; Alderbrigde et al. (1983) J. Animal Sci. 1198; Thomas and Beeson (1977) J. Animal Sci. 45:819; Morris et al. (1973) Poultry Science 52:858; Moran et al. (1967) Poultry Science 46:456; Davis et al. (1961) Processing of poultry by-products and their utilization in feeds, Part I. USDA Util. Res. Rep. no. 3, Washington, D.C.

Disadvantages of these procedures, such as degradation of heat-sensitive amino acids by steam processes and the relatively low digestibility of the resulting products, have led to continued interest in new, more economical feather degradation procedures that do not require a harsh steam treatment. A solution to the foregoing problem is reported in U.S. Pat. No. 4,908,220 to Shih et al, the disclosure of which is incorporated by reference herein in its entirety. This patent describes a hydrolyzed feather animal feed supplement consisting of feather hydrolyzed by fermenting it with Bacillus licheniformis PWD-1 prior to feeding the material to the animal. Moreover, U.S. Pat. No. 5,712,147 to Shih et al. describes DNA encoding Bacillus Licheniformis PWD-1 keratinase. While the technology disclosed in these applications provide a process in which to substantially increase the digestibility of feather, a fermentation step is necessary (e.g. such as is described in U.S. Pat. Nos. 4,959,311 and 5,063,161, both to Shih et al., the disclosures of which are incorporated by reference herein in their entirety) that adds to the complexity of manufacturing the feed.

Immobilized proteases and peptidases can perform complete hydrolysis of protein to amino acids. See e.g., Church FC, et al (1984) J. Appl. Biochem 6: 205-211; Swaisgood HE, et al., (1989) ACS Symposium Series 389, ed. JRWaPES (eds.). Washington, D.C: American Chemical Society. 242-261. Immobilized proteases can also be used to probe protein structure (See e.g., Burgess AW, et al. (1975) Biochem, 28: 5421-5428; Church, FC et al. (1982) Enzyme Microb. Technol. 4: 317-321) and to release protein domains (See e.g., Girma JP, et al. (1986) Biochem., 25: 3156-3163; Swaisgood HE, et al., (1994) Protein Structure-Function Relationship in Foods, ed. RLJaJLSe R. Y. Yada: Blackie Academic & professional, Glascow. 43-61). In nutrition, an immobilized digestive enzyme assay has been developed to evaluate protein digestibility to mimic the digestion in the stomach and intestine system. See e.g, Porter DH, et al. (1984) Agr Food Chem 32: 334-339; Swaisgood HE, et al. Advances in Food and Nutrition Research, ed. JEK (ed.). Vol. 35. 1991, London.: Elsevier Applied Science Publishers. 309-341. Chemical immobilization of proteases on a solid matrix has been demonstrated with trypsin (Chen SX, et al. (1994) Journal of Agriculture and Food Chemistry, 42: 234-239), subtilisin (Chapman JD, et al. (1975) Biotechnol. Bioeng., 17: 1783-1795; Nishio T, et al. (1984) Archives of Biochemistry and Biophysics, 229: 304-311), and keratinase (Lin X, et al. (1996) Appl. Env. Microb., 62: 4273-4275). Chemically immobilized enzymes involve covalent binding, thus has longer lifetimes than can be achieved with methods of physical immobilization such as adsorption. However, the enzyme activity and substrate binding capacity often decrease due to the steric effect of random immobilization. Protein engineering followed by chemical immobilization can improve the subtilisin activity by site-specific immobilization. See e.g., Huang W, et al. (1997) Anal. Chem. 69: 4601-4607; Viswanath S, et al. (1998) Biotechnol Bioeng, 60: 608-616. The oriented immobilized subtilisin has higher catalytic efficiency. Nevertheless, applications of chemically immobilized enzymes are limited because, prior to chemical immobilization, the enzyme must be purified. This process is laborious and costly.

It is desirable to provide a process for hydrolyzing proteinaceous or keratinaceous material that does not depend on steam hydrolysis and/or increases the digestibility if keratin without the necessity of fermenting the material prior to feeding.

### SUMMARY OF THE INVENTION

A first aspect of the present invention is a recombinant nucleic acid encoding a fusion protein, said recombinant nucleic acid comprising a nucleic acid encoding a protein (preferably an enzyme such as a proteinase, and more preferably a keratinase) fused to nucleic acid encoding a first member of a specific binding pair. The nucleic acid encoding a keratinase may, for example, be (a) nucleic acid encoding the Bacillus licheniformis PWD-1 keratinase; (b) nucleic acid that hybridizes to a nucleic acid of (a) mentioned previously under stringent conditions (for example, conditions represented by a wash stringency of 0.3 M NaCl, 0.03 M sodium citrate, 0.1% SDS at 70° C.); and (c) nucleic acid that differs from the nucleic acid of (a) and (b) mentioned earlier due to the degeneracy of the genetic code, and which encodes a protein encoded by the nucleic acids of (a) and (b) mentioned previously. In particular embodiments, the nucleic acid encoding a keratinase encodes the Bacillus licheniformis PWD-1 keratinase, or encodes the Bacillus licheniformis NCIB 6816 subtilisin Carlsberg serine protease.

Preferred specific binding pairs for carrying out the present invention include (a) antigens and antibodies, and (b) biotin and avidin. In one embodiment, the first member of a specific binding pair is avidin (streptavidin).

A second aspect of the present invention is an expression vector such as a plasmid comprising a nucleic acid encoding a fusion protein as described above operably associated with a promoter.

A third aspect of the present invention is a host cell that contains an expression vector as described above and expresses the encoded fusion protein therein. Preferred host cells include, but are not limited to, Bacillis subtilis or Escherichia coli, and more preferably Bacillus subtilis because of its secretion of the fusion protein.

A fourth aspect of the present invention is a method of making a fusion protein, comprising: (a) providing a host cell as described above,(b) expressing the encoded fusion protein in the host cell; and then (c) collecting the encoded protein. In one embodiment, the encoded protein is secreted by said host cell. The collecting step may be carried out by contacting the encoded protein to a solid support, said solid support having a second member of said binding pair bound thereto, to which the first member of the binding pair specifically binds (e.g., biotin, when the first member is avidin).

A fifth aspect of the present invention is a fusion protein comprising a keratinase fused to a first member of a specific binding pair. The fusion protein may be encoded by a nucleic acid as described above.

A sixth aspect of the present invention is an immobilized keratinase comprising: (a) a fusion protein as described above; and (b) a solid support such as a bead, the solid support having a second member of said specific binding pair bound thereto; wherein the first member of said specific binding pair (e.g., avidin) is bound to the second member of the specific binding pair (e.g., biotin).

A seventh aspect of the present invention is a method of digesting a substrate such as keratin or protein (e.g., for producing protein fragments, peptides, amino acids), comprising: (a) providing an immobilized keratinase as described above, and then (b) contacting (continuously or in a batch process) a substrate such as protein or keratin to the immobilized keratin for a time sufficient to at least partially digest the substrate.

An eighth aspect of the invention is a method of digesting protein, keratin, or casein further comprising the step of collecting the degradation product.

The foregoing and other objects and aspects of the present invention are explained in greater detail in the drawings herein and the specification set forth below.

### BRIEF DESCRIPTION OF THE DRAWINGS

FIG. 1 illustrates fusion constructs carrying the keratinase-strepavidin fusion gene expressed in B. subtilis.

FIG. 2 illustrates construction of plasmids harboring the keratinase and keratinase-streptavidin fusion genes including pro- and prepro-regions expressed in E. coli. Legend: pelB (57 bp), leader sequence; [symbol] kerA (840bp), which corresponds to mature keratinase without pre- and pro-; Pre-(78 bp); Pro-(234bp); stp (496bp), full length of strepavidin gene; stpc (360bp), core barrel of stp.

FIG. 3 illustrates construction of plasmids for the expression of keratinase-streptavidin fusion protein in B. subtilis DB104 or WB600.

FIG. 4 illustrates construction of plasmids for the expression of keratinase and keratinase-streptavidin fusion proteins in E. coli.

FIG. 5 illustrates identification of fusion gene expression in Bacillus media by SDS PAGE Each lane was loaded 0.5 mL supernatant. Lane M: protein marker. Lanes 2-5: supernatant at 12, 24, 36, and 48 h culture from pJC/DB104.

FIG. 6 illustrates fusion proteins from B. subtilis analyzed by Western blot. Lanes 2 and 3 were loaded with 50 μg of total protein. Lane 1: pure streptavidin (Sigma). Lane 2: culture supernatant at 16 h from pJC/WB600. Lane 3: culture supernatant at 16 h from pJCD/WB600. dSTP: dimeric STP. mSTP: mono STP.

FIG. 7 illustrates SDS-PAGE analysis of overexpression of keratinase and keratinase-streptavidin fusion proteins from E. coli BL21 (DE3) pLysS. Lane M: molecular marker; 1: pure keratinase from B. licheniformis PWD-1 (31 kDa); 2: total cellular protein from pKER (31 kDa); 3: total cellular protein from pProK(42 kDa); 4: total cellular protein from pKSTP (48 kDa); 5: total cellular protein from pProKSTP (58 kDa); 6: total cellular protein from pKSTPC (42 kDa); 7: total cellular protein from pProKSTPC (48 kDa).

FIG. 8 illustrates western blot analysis of fusion protein produced from E. coli with anti-strepavidin antibody and anti-keratinase antiserum. Lane A: total cellular protein from pKSTP/E coli BL21(DE3) pLysS; B: total cellular protein from pKSTPC/E. coli BL21(DE3) pLysS; C: streptavidin control, dSTP:dimer, mSTP:monomer.

FIG. 9 illustrates immobilization of keratinase-streptavidin fusion protein on biotinylated beads. Lane M: molecular marker; Lane 1: total cellular protein from pKSTP/BL21(DE3) pLysS. Lane 2: total cellular protein from pProKSTP/BL21 (DE3) pLysS. Lane 3: periplasmic and cytoplasmic proteins. Lane 4: pro-keratinase-streptavidin inclusion body (58 kDa). Lane 5: inclusion body after dialysis with agarose biotin beads against 200 mM Na₂HPO₄ at pH 7.0 at 4° C. overnight. Lane 6: inclusion body after dialysis with acrylic biotin beads against 200 mM Na₂HPO₄ at pH 7.0 at 4° C. overnight.

FIG. 10 illustrates Caseinolytic activity of soluble and immobilized keratinase pretreated at different pH.

FIG. 11 illustrates pH-activity profile of soluble and immobilized keratinase against azocasein and azokeratin as substrate.

FIG. 12 illustrates stability and durability of free and immobilized keratinase. Enzyme activity was measured by azocasein hydrolysis.

FIG. 13 illustrates the increase of free amino groups during digestion of casein and feather keratin by immobilized keratinase-streptavidin. Free amino acid group release was measured by ninhydrin method with leucine equivalent as standard.

### DETAILED DESCRIPTION OF THE PREFERRED EMBODIMENTS

The present invention will now be described more fully hereinafter with reference to the accompanying figures, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

The present invention can be carried out with all types of keratinaceous material, including hair, hooves, and feather. Feather is preferred. Any type of feather may be employed, including chicken, turkey, and duck feather. Chicken feather is preferred, and is the material recited in the text below. However, teaching of this text is applicable to the degradation and utilization of all keratinaceous materials. As used herein, substances suitable for degradation by keratinases include, but are not limited to, keratin, collagen, elastin, and proteins such as casein and bovine serum albumin, and gelatin.

Amino acid sequences disclosed herein are presented in the amino to carboxy direction, from left to right. The amino and carboxy groups are not presented in the sequence. Nucleotide sequences are presented herein by single strand only, in the 5′ to 3′ direction, from left to right. Nucleotides and amino acids are represented herein in the manner recommended by the IUPAC-IUB Biochemical Nomenclature Commission, or (for amino acids) by three-letter code, in accordance with 37 C.F.R §1.822 and established usage. See, e.g., Patent In User Manual, 99-102 (Nov. 1990) (U.S. Patent and Trademark Office).

The disclosures of all United States patent references cited herein are to be incorporated by reference herein in their entirety.

Suitable nucleic acid sequences encoding a keratinase are given in U.S. Pat. No. 5,712,147 to Shih et al., the disclosure of which is incorporated herein by reference. SEQ ID NO: 1-2 herein are intended to correspond to SEQ ID NO: 1-2 therein.

“Nucleic acid sequence” as used herein refers to an oligonucleotide, nucleotide, or polynucleotide, and to DNA or RNA of genomic or synthetic origin which may be single- or double-stranded, and represent the sense or antisense strand.

Polynucleotides of the present invention include those coding for proteins homologous to, and having essentially the same biological properties as, the proteins disclosed herein, and particularly the DNA disclosed herein as SEQ ID NO: 1. This definition is intended to encompass natural allelic sequences thereof. Thus, isolated DNA or cloned genes of the present invention can be of any species of origin, but various strains of Bacillus subtilis are currently preferred. Thus, polynucleotides that hybridize to DNA disclosed herein as SEQ ID NO: 1 and which code on expression for a keratinase, are also an aspect of the invention. Conditions which will permit other polynucleotides that code on expression for a protein of the present invention to hybridize to the DNA of SEQ ID NO: 1 herein can be determined in accordance with known techniques.

For example, hybridization of such sequences may be carried out under conditions of reduced stringency, medium stringency or even stringent conditions (e.g., conditions represented by a wash stringency of 35-40% Formamide with 5×Denhardt's solution, 0.5% SDS and 1×SSPE at 37° C.; conditions represented by a wash stringency of 40-45% Formamide with 5×Denhardt's solution, 0.5% SDS, and 1×SSPE at 42° C.; and conditions represented by a wash stringency of 50% Formamide with 5×Denhardt's solution, 0.5% SDS and 1×SSPE at 42° C., respectively) to DNA of SEQ ID NO: 1 herein in a standard hybridization assay. See, e.g., J. Sambrook et al., Molecular Cloning, A Laboratory Manual (2d Ed. 1989) (Cold Spring Harbor Laboratory). In general, sequences which code for proteins of the present invention and which hybridize to the DNA of SEQ ID NO: 1 disclosed herein will be at least 75% homologous, 85% homologous, and even 95% homologous or more with SEQ ID NO: 1. Further, polynucleotides that code for proteins of the present invention, or polynucleotides that hybridize to that as SEQ ID NO: 1, but which differ in codon sequence from SEQ ID NO: 1 due to the degeneracy of the genetic code, are also an aspect of this invention. The degeneracy of the genetic code, which allows different nucleic acid sequences to code for the same protein or peptide, is well known in the literature. See, e.g., U.S. Pat. No. 4,757,006 to Toole et al. at Col. 2, Table 1.

The production of cloned genes, recombinant DNA, vectors, transformed host cells, proteins and protein fragments by genetic engineering is well known. See, e.g., U.S. Pat. No. 4,761,371 to Bell et al. at Col. 6 line 3 to Col. 9 line 65; U.S. Pat. No. 4,877,729 to Clark et al. at Col. 4 line 38 to Col. 7 line 6; U.S. Pat. No. 4,912,038 to Schilling at Col. 3 line 26 to Col. 14 line 12; and U.S. Pat. No. 4,879,224 to Wallner at Col. 6 line 8 to Col. 8 line 59.

A vector is a replicable DNA construct. Vectors are used herein either to amplify DNA encoding the proteins of the present invention or to express the proteins of the present invention. An expression vector is a replicable DNA construct in which a DNA sequence encoding the proteins of the present invention is operably linked to suitable control sequences capable of effecting the expression of proteins of the present invention in a suitable host. The need for such control sequences will vary depending upon the host selected and the transformation method chosen. Generally, control sequences include a transcriptional promoter, an optional operator sequence to control transcription, a sequence encoding suitable mRNA ribosomal binding sites, and sequences which control the termination of transcription and translation. Amplification vectors do not require expression control domains. All that is needed is the ability to replicate in a host, usually conferred by an origin of replication, and a selection gene to facilitate recognition of transformants.

Vectors comprise plasmids, viruses (e.g., adenovirus, cytomegalovirus), phage, retroviruses and integratable DNA fragments (i.e., fragments integratable into the host genome by recombination). The vector replicates and functions independently of the host genome, or may, in some instances, integrate into the genome itself. Expression vectors should contain a promoter and RNA binding sites which are operably linked to the gene to be expressed and are operable in the host organism.

DNA regions are operably linked or operably associated when they are functionally related to each other. For example, a promoter is operably linked to a coding sequence if it controls the transcription of the sequence; a ribosome binding site is operably linked to a coding sequence if it is positioned so as to permit translation. Generally, operably linked means contiguous and, in the case of leader sequences, contiguous and in reading phase.

Transformed host cells are cells which have been transformed or transfected with vectors containing DNA coding for proteins of the present invention and need not express protein. However, in the present invention, the cells preferably express the protein, and more preferably secret the encoded protein.

Suitable host cells include prokaryotes, yeast cells, or higher eukaryotic organism cells. Prokaryote host cells include gram negative or gram positive organisms, for example Escherichia coli (E. coli) or Bacilli. Bacillus subtilis is particularly preferred. Higher eukaryotic cells include established cell lines of mammalian origin as described below. Exemplary host cells are E. coil W3110 (ATCC 27,325), E. coli B, E. coli X1776 (ATCC 31,537), E. coli 294 (ATCC 31,446). A broad variety of suitable prokaryotic and microbial vectors are available. E. coli is typically transformed using pBR322. See Bolivar et al., Gene 2, 95 (1977). Promoters most commonly used in recombinant microbial expression vectors include the beta-lactamase (penicillinase) and lactose promoter systems (Chang et al., Nature 275, 615 (1978); and Goeddel et al., Nature 281, 544 (1979), a tryptophan (trp) promoter system (Goeddel et al., Nucleic Acids Res. 8, 4057 (1980) and EPO App. Publ. No. 36,776) and the tac promoter (H. De Boer et al., Proc. Natl. Acad. Sci. USA 80, 21 (1983). The promoter and Shine-Dalgarno sequence (for prokaryotic host expression) are operably linked to the DNA of the present invention, i.e., they are positioned so as to promote transcription of the messenger RNA from the DNA.

Expression vectors should contain a promoter which is recognized by the host organism. This generally means a promoter obtained from the intended host. Promoters most commonly used in recombinant microbial expression vectors include the beta-lactamase (penicillinase) and lactose promoter systems (Chang et al., Nature 275, 615 (1978); and Goeddel et al., Nature 281, 544 (1979), a tryptophan (trp) promoter system (Goeddel et al., Nucleic Acids Res. 8, 4057 (1980) and EPO App. Publ. No. 36,776) and the tac promoter (H. De Boer et al., Proc. Natl. Acad. Sci. USA 80, 21 (1983). While these are commonly used, other microbial promoters are suitable. Details concerning nucleotide sequences of many have been published, enabling a skilled worker to operably ligate them to DNA encoding the protein in plasmid or viral vectors (Siebenlist et al., Cell 20, 269 (1980). The promoter and Shine-Dalgarno sequence (for prokaryotic host expression) are operably linked to the DNA encoding the desired protein, i.e., they are positioned so as to promote transcription of the protein messenger RNA from the DNA.

Eukaryotic microbes such as yeast cultures may be transformed with suitable protein-encoding vectors. See e.g., U.S. Pat. No. 4,745,057. Saccharomyces cerevisiae is the most commonly used among lower eukaryotic host microorganisms, although a number of other strains are commonly available. Yeast vectors may contain an origin of replication from the 2 micron yeast plasmid or an autonomously replicating sequence (ARS), a promoter, DNA encoding the desired protein, sequences for polyadenylation and transcription termination, and a selection gene. An exemplary plasmid is YRp7, (Stinchcomb et al., Nature 282, 39 (1979); Kingsman et al., Gene 7, 141 (1979); Tschemper et al., Gene 10, 157 (1980). This plasmid contains the trp1 gene, which provides a selection marker for a mutant strain of yeast lacking the ability to grow in tryptophan, for example ATCC No. 44076 or PEP4-1 (Jones, Genetics 85, 12 (1977). The presence of the trp1 lesion in the yeast host cell genome then provides an effective environment for detecting transformation by growth in the absence of tryptophan.

Suitable promoting sequences in yeast vectors include the promoters for metallothionein, 3-phospho-glycerate kinase (Hitzeman et al., J. Biol. Chem. 255, 2073 (1980) or other glycolytic enzymes (Hess et al., J. Adv. Enzyme Reg. 7, 149 (1968); and Holland et al., Biochemistry 17, 4900 (1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Suitable vectors and promoters for use in yeast expression are further described in R. Hitzeman et al., EPO Publn. No. 73,657.

Cultures of cells derived from multicellular organisms are a desirable host for recombinant protein synthesis. In principal, any higher eukaryotic cell culture is workable, whether from vertebrate or invertebrate culture, including insect cells. Propagation of such cells in cell culture has become a routine procedure. See Tissue Culture, Academic Press, Kruse and Patterson, editors (1973). Examples of useful host cell lines are VERO and HeLa cells, Chinese hamster ovary (CHO) cell lines, and WI138, BHK, COS-7, CV, and MDCK cell lines. Expression vectors for such cells ordinarily include (if necessary) an origin of replication, a promoter located upstream from the gene to be expressed, along with a ribosome binding site, RNA splice site (if intron-containing genomic DNA is used), a polyadenylation site, and a transcriptional termination sequence.

The transcriptional and translational control sequences in expression vectors to be used in transforming vertebrate cells are often provided by viral sources. For example, commonly used promoters are derived from polyoma, Adenovirus 2, and Simian Virus 40 (SV40). See, e.g., U.S. Pat. No. 4,599,308. The early and late promoters are useful because both are obtained easily from the virus as a fragment which also contains the SV40 viral origin of replication. See Fiers et al., Nature 273, 113 (1978). Further, the protein promoter, control and/or signal sequences, may also be used, provided such control sequences are compatible with the host cell chosen.

An origin of replication may be provided either by construction of the vector to include an exogenous origin, such as may be derived from SV40 or other viral source (e.g. Polyoma, Adenovirus, VSV, or BPV), or may be provided by the host cell chromosomal replication mechanism. If the vector is integrated into the host cell chromosome, the latter may be sufficient.

Host cells such as insect cells (e.g., cultured Spodoptera frugiperda cells) and expression vectors such as the baculorivus expression vector (e.g., vectors derived from Autographa californica MNPV, Trichoplusia ni MNPV, Rachiplusia ou MNPV, or Galleria ou MNPV) may be employed to make proteins useful in carrying out the present invention, as described in U.S. Pat. Nos. 4,745,051 and 4,879,236 to Smith et al. In general, a baculovirus expression vector comprises a baculovirus genome containing the gene to be expressed inserted into the polyhedrin gene at a position ranging from the polyhedrin transcriptional start signal to the ATG start site and under the transcriptional control of a baculovirus polyhedrin promoter.

Host cells transformed with nucleotide sequences encoding a protein or peptide of the invention may be cultured under conditions suitable for the expression and recovery of the protein from cell culture. The protein produced by a transformed cell may be secreted or contained intracellularly depending on the sequence and/or the vector used. As will be understood by those of skill in the art, expression vectors containing polynucleotides which encode a protein or peptide of the invention may be designed to contain signal sequences which direct secretion of the protein or peptide through a prokaryotic or eukaryotic cell membrane. Other constructions may be used to join sequences encoding the protein or peptide to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the protein or peptide of the invention may be used to facilitate purification. One such expression vector provides for expression of a fusion protein containing The protein or peptide of the invention and a nucleic acid encoding 6 histidine residues preceding a thioredoxin or an enterokinase cleavage site. The histidine residues facilitate purification on IMAC (immobilized metal ion affinity chromatography) as described in Porath, J. et al. (1992, Prot. Exp. Purif 3: 263-281) while the enterokinase cleavage site provides a means for purifying the protein or peptide of the invention from the fusion protein. A discussion of vectors which contain fusion proteins is provided in Kroll, D. J. et al. (1993; DNA Cell Biol. 12:441-453).

Any type of solid support may be used to carry out the present invention, including beads, particles, rods, and other shapes, formed of any suitable material such as glass, ceramic, polymer, gel, etc. The fusion protein may be immobilized to the solid support with or without intervening processing steps, such as cell lysis (it being appreciated that cell lysis is required when the fusion protein is not secreted by the host cell).

In a preferred embodiment of the invention, isolation and immobilization of the fusion protein is achieved in a single step by mixing the solid support with a growth medium, preferably a liquid growth medium, in which the host cells have been grown and into which the fusion protein has been secreted so that the first and second members of the specific binding pair then bind to one another. Cell lysis can, if necessary, be carried out in the growth medium, although the method is particularly simple when the fusion protein is secreted and no cell lysis is required. The solid support can then be easily separated from the growth medium in accordance with known techniques.

Once the fusion protein is immobilized to the solid support, digestion of the protein or keratin (e.g., feathers, casein) can be carried out in accordance with known techniques or variations thereof that will be apparent to those skilled in the art. Degradation products from partial or complete digestion of the protein or keratin can then be collected (if desired) in accordance with standard techniques. The invention is useful for, among other things, the production of a feather lysate as described in U.S. Pat. Nos. 4,908,220; 4,959,311; 5,063,161; 5,171,682; and 5,186,961, all to Shih et al.

While the present invention has been described primarily with reference to a keratinase, it will be appreciated that other proteins of interest, particularly other enzymes such as other proteases, can be substituted for the keratinase in the fusion proteins described herein.

### EXAMPLES

### Example 1

### Bacterial strains, plasmids, and growth conditions.

Bacillus licheniformis PWD-1 (ATCC 53575, Williams et al. (1990) Appl. Env. Microb. 56:1509-1515) was used to isolate the kerA gene. Bacillus subtilis DB104 (his nprR2 nprE18 aprΔ3) (Kawamura and Doi, (1984) J. Bacteriol. 160:442-444), WB600 (trpC2 nprA apr epr bfp mpr:ble nprB::ery) (Wu et al. (1991) J. Bacteriol. 173:4952-4958.), Escherichia coli Novoblue, BL21(DE3) (F⁻ompT HsdS_{B} gal dcm (DE3)) (Studier and Moffatt, (1986) J. Mol. Biol. 189:113-130.) and BL21(DE3) pLysS (F⁻ompT HsdS_{B} gal dcm (DE3) pLysS) (Studier et al. (1990) Methods Enzymol. 185:60-69.) were used as hosts for cloning and expression studies. The E. coli plasmid pETSA7, containing the full length of strepavidin gene, was used as previously described (Walsh and Swaisgood, (1994) Biotech. Bioeng. 44:1348-1354). Plasmids pUB18-P43 carrying the P43 promoter was used for insertion of the fusion genes in B. subtilis (Wang and Doi, (1987) Mol. Gen. Genet. 207:114-119). Vector pET-26b(+) (Novagen, Madison Wis.), containing the T7 promoter, pelB leader sequence and His-Tag was used in E. coli. PWD-1 was grown in either feather or Luria-Bertani (LB) medium at 50° C. B. subtilis and E. coli strains were grown at 37° C. in LB medium containing 20 mg/mL kanamycin for routine transformation and gene expression.

### Example 2

### DNA manipulation.

The mini-preparations of plasmids from Bacilli were made by the rapid alkaline sodium dodecyl sulfate methods (Rodriguez and Tait, (1983) Recombinant DNA techniques. Addison-Wesley Publishing Co., Inc., Reading, Mass.). Isolation of plasmids from E. coli was performed according to standard protocols (Sambrook et al. (1989) Molecular cloning: A Laboratory Manual, 2nd Ed. Cold Spring Harbor Laboratory Press, Cold Spring Harbor, N.Y.). Chromosomal or genomic DNA of PWD-1 was isolated using a method previously described (Doi (1983) Isolation of Bacillus subtilis chromosomal DNA. In Recombinant DNA techniques. R. L. Rodriquez and R. C. Trait, eds. Addison-Wesley Publishing Co. Reading, Mass. p. 162-163). Restriction enzymes and DNA ligases were purchased from Promega (Madison Wis.) and Boehringer-Mannheim (Mannheim, Germany) and used as recommended by the manufacturers. PCR reactions were performed with either pfu (Boehringer-Mannheim) or Taq (Promega) DNA polymerase by the following conditions: 94° C. for 1 min, 56° C. for 1.5 min, 72° C. for 2 min. (30 cycles) and 72° C. for 5 min. DNA fragments were separated by 0.8 to 1.2% agarose gel. The desired DNA fragment and PCR products were recovered and purified by the QIAquick Gel Extraction Kit and PCR Purification Kit (Qiagen Inc, Valencia, Calif.) respectively.

### Example 3

### Isolation and amplification of kera and streptavidin (stp) genes.

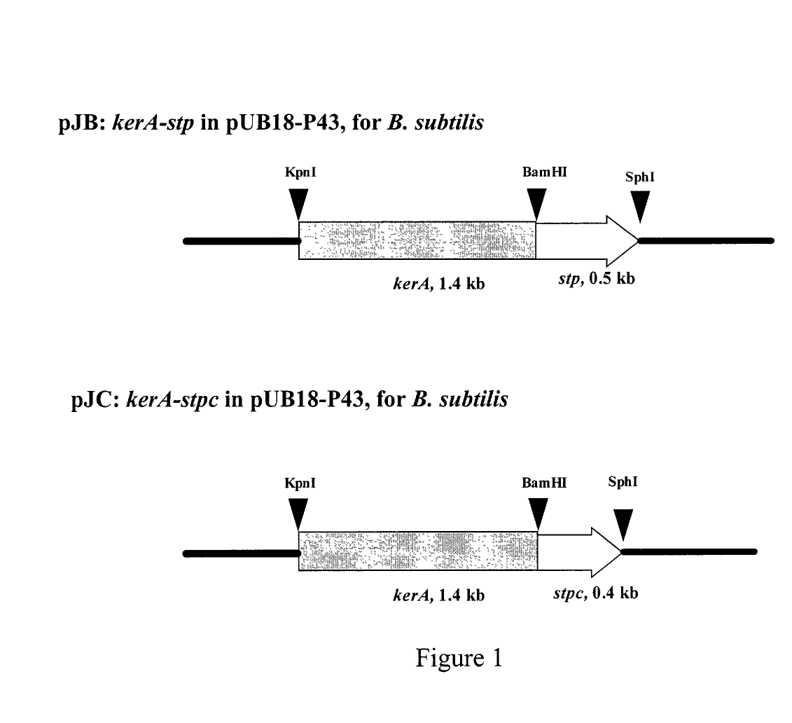

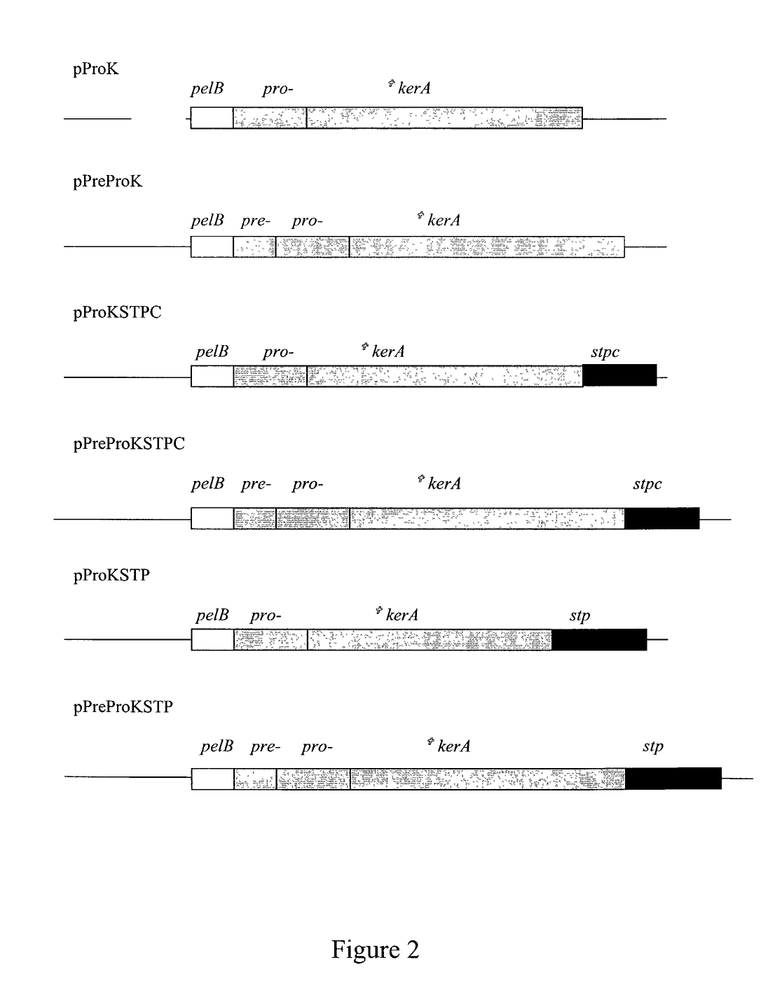

All specifically designed primer sequences are listed in Table 1. They were used for PCR in producing DNA sequences with desirable restriction sites and mutated codons for the construction of modified fusion genes in different plasmids (FIGS. 1 and 2). Gene kerA (1.4 kbp) from PWD-1 genomic DNA and streptavidin gene (stp, 0.5 kbp) from pETSA7/E. coli were amplified by PCR. Primers containing unique restriction sites as well as mutated START or STOP codons were used (Table 1).

In the Bacillus system, the full length of kerA, including the promoter, pre- and pro-regions, was amplified and cloned into pCR2.1 vector (Novagen), creating the pCRKER plasmid. The 3′ primer (KERBamHI, SEQ ID NO: 3) mutated the STOP codon and created a BamHI restriction site at the end of kerA gene for fusion in-frame with stp. Similarly, the 5′ end of stp was modified by PCR to introduce a unique BamHI site for cloning in-frame to the 3′ end of kerA and for creation of a STOP codon at the 3′ end. The stp gene was then inserted into pCR2.1. The gene constructs are shown in FIG. 1.

The same technique was used in E. coli systems to isolate kerA and stp except that different primers were used (Table 1). The 5′ primer (KERNcoI, SEQ ID NO: 9)

TABLE 1
Primers used for the construction of plasmids
harboring keratinase and keratinase-strepavidin
fusion genes.

| | |
|---|---|
| Primer | Nucleotide sequence |

(SEQ ID NO:3)

| | |
|---|---|
| KERKpnI | CGAACGGGGTACCCTCCTGCCAAGCTGAAGCGGTCT |

(SEQ ID NO:4)

| | |
|---|---|
| KERBamHI | CGCGGATCCTGAGCGGCAGCTTCGAC |

(SEQ ID NO:5)

| | |
|---|---|
| STPBamHI | CGCGGATCCCTCCAAGGACTCGAAGG |

(SEQ ID NO:6)

| | |
|---|---|
| STPCBamHI | ACGCACGCGGATCCCGGCATCACCGGCACCTGGTACAAC |

(SEQ ID NO:7)

| | |
|---|---|
| STPSphI | ACATACATGCATGCGAGCTCTACTGCTGAACGGCGTCGA |

G
(SEQ ID NO:8)

| | |
|---|---|
| STPCSphI | CACATACATGCATGCTTACGGCTTACACTTGGTGAAGGT |

(SEQ ID NO:9)

| | |
|---|---|
| KERNcoI | CATGCCATGGCGCAAACCGTTCCTTAC |

(SEQ ID NO:10)

| | |
|---|---|
| KERXhoI | CCGCTCGAGTTGAGCGGCAGCTTCGAC |

(SEQ ID NO:11)

| | |
|---|---|
| KERDELBamHI | CGCACGCGGATCCTCGACATTGATCAGACCTTTCCC |
| ProKERNcoI | TCAGCATGCCATGGCTGCTCAACCGGCGAAA |

created a new start ATG codon with a NcoI restriction site and removed the pre- and pro-sequences, i.e. only the mature protein sequence (840 bp) was amplified. The STOP codon at the end of kerA gene was mutated with a unique BamHI site suitable for cloning in-frame with the 5′ end of stp. To test the function of pro- and prepro-regions in the refolding of the fusion protein, primers ProKERNcoI (SEQ ID NO: 12) and PreProKERNcoI (SEQ ID NO: 13) were used to generate constructs as shown in FIG. 2.

### Example 4

### Construction of kerA-stp vectors.

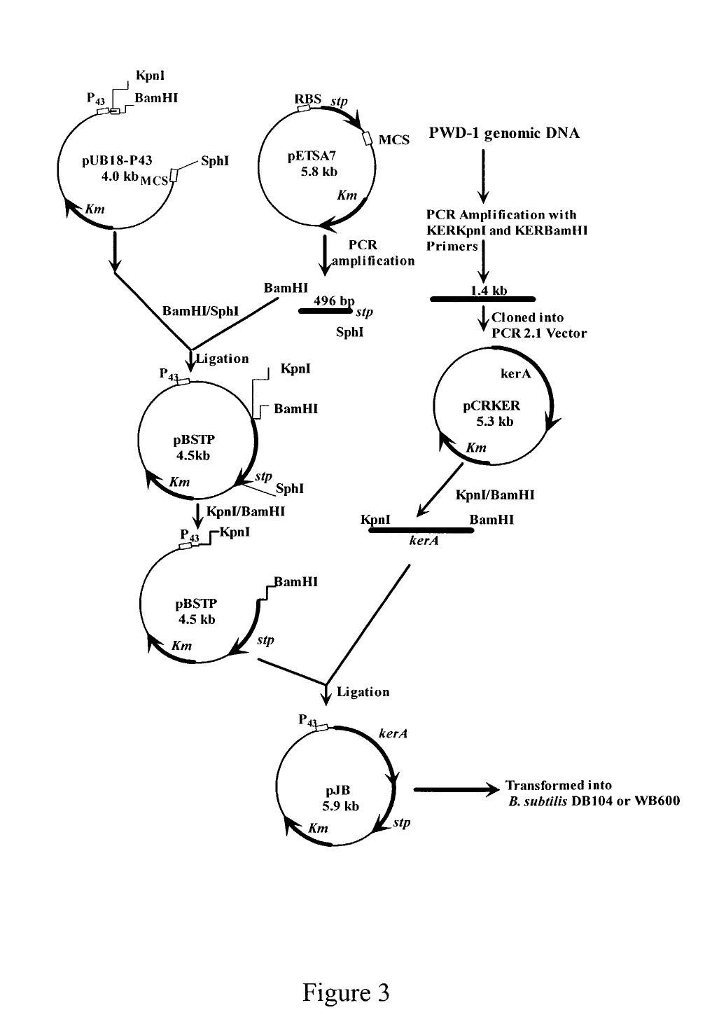

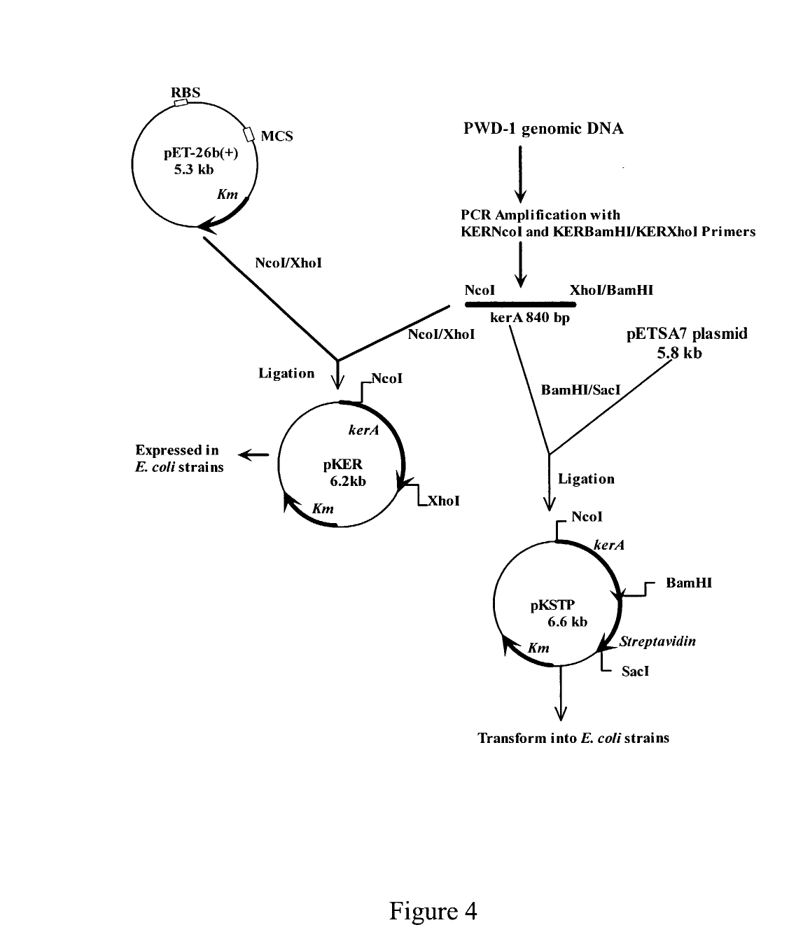

For the Bacillus system (FIG. 3), the 496-bp stp PCR product was cleaved by BamHI and SphI and ligated into pUB18-P43 digested the same way, creating the pUBSTP plasmid. The gene kerA isolated from pCRKER by cleavages with KpnI and BamHI was subcloned in-frame into similarly digested pUBSTP, generating pJB. For the E. coli system (FIG. 4), the 840-bp PCR product, amplified from KERNcoI (SEQ ID NO: 9) and KERXhoI (SEQ ID NO: 10) primers, cleaved with NcoI and XhoI, and ligated to similarly digested pET-26b(+) to create a new plasmid vector pKER. Subsequently, this new vector was introduced into two different E. coli strains to test for the expression of kerA. The other 840 base-pair kerA PCR product, amplified with primers KERNcoI (SEQ ID NO: 9) and KERBamHI (SEQ ID NO: 4), cleaved with NcoI and BamHI and ligated in-frame to the similarly digested pETSA7 plasmid containing stp, thereby creating a new plasmid, pKSTP. When the stp fusion gene was replaced by stpc (core streptavidin gene) in the plasmids, pJC and pSTPCK were generated. Additional constructs are shown in FIG. 2.

### Example 5

### Transformation.

B. subtilis and E. coli strains were transformed by the constructed plasmids described in Example 3. Transformation of B. subtilis DB104 and WB600 was carried out as previously described (Lin et. al, (1997) J. Ind. Microb. Biotech. 19:134-138). Calcium chloride transformation of E. coli was performed according to known methods (Sambrook et al. (1989) Molecular cloning: A Laboratory Manual, 2nd Ed. Cold Spring Harbor Laboratory Press, Cold Spring Harbor, N.Y.). Transformants were selected on LB plates containing 20 μg/mL kanamycin. Gene insertion was confirmed by restriction digestion and PCR amplification of isolated plasmids.

### Example 6

### DNA sequence analysis.

The fusion gene inserted in the expression vector was confirmed by DNA sequencing. Pure concentrated plasmids harboring the fusion gene were prepared and resolved on the ABI Prism 377 sequencer (North Carolina State University). In addition to primers listed in Table 1, internal primers were used to obtain overlapping regions to confirm sequence data. All sequence data were analyzed by GCG Wisconsin Package (Madison, Wis.).

### Example 7

### Enzyme assay and protein determination.

Keratinase activity was measured by azokeratin hydrolysis as described previously (Lin et al. (1992) Appl. Env. Microb. 58:3271-3275) and the protein concentration was determined by the Bio-Rad Microassay procedure (Bradford, (1976) Anal. Biochem. 72:248-254). The immobilized protein was measured by the OPA (o-phthaldialdehyde) assay (Church et al. (1982) Enzyme Microb. Technol. 4:317-321; Thresher (1989) Characterization of macromolecular interactions by high performance analytical affinity chromatography. Ph.D. dissertation, North Carolina State University, Raleigh, N.C.).

### Example 8

### Expression of the KER-STP fusion protein in B. subtilis.

Expression of keratinase-streptavidin (KER-STP) fusion protein was determined by both RNA and protein analyses. Messenger RNA of the fusion gene was determined by RNA dot blot as described previously (Wang and Shih, 1999). Digoxigenin-labeled probes for the detection of streptavidin gene were amplified from pETSA7 in E. coli by PCR using the PCR DIG Labeling mix (Boehringer-Mannheim, Mannheim, Germany). Primers STPCBamHI and STPCSphI (Table 1) were used to amplify a digoxigenin-labeled 360 bp stpc.

KER-STP was produced extracellularly as previously described (Lin et al. (1992) Appl. Env. Microb. 58:3271-3275). Briefly, the culture media of B. subtilis transformants was collected and assayed for proteolytic and keratinolytic activities. Precipitated by 5% TCA, concentrated proteins were analyzed by sodium dodecyl sulfate polyacrylamide gel electrophoresis (SDS-PAGE) (Laemmli, (1970) Nature 227:680-685). Western blotting was modified as described by Towbin et al. (1979) Proc. Natl. Acad. Sci. USA 76:4350-4354. From SDS-PAGE, proteins were transferred to a nitrocellulose membrane and probed with either anti-streptavidin (Chemicon International Inc., CA) or anti-keratinase rabbit antiserum.

### Example 9

### Extraction of KER-STP protein in E. coli.

The culture of E. coli was induced by the addition of 0.1 mM isopropylthiogalactoside (IPTG) and incubation for 2-4 hr. After induction, cells were harvested by centrifugation. Three fractions of proteins, periplasmic, cytoplasmic, and insoluble inclusion body, were separated. The fraction of periplasmic protein was extracted using osmotic shock in 20% sucrose (Sprott et al. (1994) Cell fractionation, in Methods for general and molecular bacteriology. American Society for Microbiology. P. Gerhardt, R. G. E. Murray, W. A. Wood and N. R. Krieg, eds. Washington, D.C. p. 72-103), then were sonically disrupted as previously described (Andrew et al. (1996) Mol. Biotech. 6:53-64; Thatcher et al. (1996) Inclusion bodies and refolding, in Proteins Labfax. N. Price, ed. BIOS Scientific, Oxford, England. p. 119-130). The cell lysate was centrifuged at 8000×g at 4° C. for 10 min. to yield the soluble cytoplasmic proteins and the insoluble inclusion bodies.

### Example 10

### Solubilization and refolding of E. coli KER-STP.

Inclusion bodies were solubilized in 6N guanidine hydrochloride in 50 mM Tris-HCl buffer, pH 8.0. The solubilized protein was renatured or refolded in vitro by dialysis at 4° C. overnight against various refolding buffers (Table 3) to recover KER-STP. Keratinase activity, protein amount and SDS-PAGE were analyzed as described above in Example 6.

### Example 11

### Immobilization of KER-STP Fusion Protein.

Two types of biotinylated solid matrix, acrylic (Sigma, Madison Wis.) and 6% agarose beads (Pierce, Rockford Ill.), were used for immobilization of the KER-STP fusion protein. The protein secreted from Bacillus cells was immobilized in situ. Biotinylated beads were loaded into sterile dialysis tubing (300 kDa cut-off, Spectrum Laboratories Inc.), placed in the growth media at the beginning or after 12 hr of culture, and allowed to grow for 24 hr. At the end, the dialysis tubing was emptied to collect the beads with immobilized keratinase.

E. coli inclusion bodies were solubilized in 6N guanidine hydrochloride, mixed with biotinylated beads in a dialysis tubing (12,000 kDa exclusion limit, Sigma) and dialyzed against the refolding buffer overnight at 4° C.

Beads with immobilized keratinase were collected and washed with 0.05 M phosphate, pH 7.5, containing 0.8% NaCl. The amount of Keratinase that typically bound to the beads was subsequently determined to be 15-20 mg protein/g beads.

### Example 12

### Kinetic studies.

N-Succinyl-Ala-Ala-Pro-Phe-p-nitroanilide (AAPF, Sigma) was selected as the substrate for kinetic studies. Enzymatic reactions were carried out in 50 mM Tris-HCl buffer, pH 8.0, at 25° C. The enzyme concentration used for soluble KE was 10.3 nM; for immobilzed KE, 10.7 nM. The AAPF substrate concentration ranged from 0.1 to 0.8 mM. A recording spectrophotometer (Shimadzu UV-Vis Recording Spectrophotometer, Shimadzu Corp., Kyoto Japan) was used to measure the change of absorbance at 405 nm (ε=9600 l/mol cm). The value of K_{M}, Vₘₐₓ, and k_{cat} were determined from Lineweaver-Burk plots.

### Example 13

### Enzyme activity.

Keratinolytic and proteolytic activity were measured by three different methods. Hydrolysis of azokeratin was measured by the increased soluble azo-peptides as described previously (Lin et al. (1992) Appl. Env. Microb. 58:3271-3275). A ninhydrin method (Rosen (1957) Arch. Biochem. Biophys. 67:10-15) was used to quantitate the increase of free amino groups released from keratinolysis, using leucine equivalent as the standard. Hydrolysis of azocasein (Sarath et al. (1989) Protease assay methods. Proteolytic enzymes: a practical approach IRJBaJSB (ed.) IRL Press, Oxford. pp. 25-55) was modified and used to determine the caseinolytic activity. Briefly, 0.2 mL of enzyme aliquot was added to preincubated 0.8 mL of 50 mM potassium phosphate buffer (pH 7.5) at 37° C. containing 0.5% azocasein (Sigma). The mixture was incubated at 37° C. for 30 min followed by addition of 0.2 mL of 10% trichloroacetic acid (TCA) to stop the reaction. The supernatant of the mixture was collected by centrifugation and the increase of absorbance at 450 nm was measured.

### Example 14

### pH pretreatment, thermal stability, and durability.

Soluble and immobilized KE were pre-treated at various pHs. For the low pH pre-treatment, 1.5 μg of soluble KE and 20 μg of immobilized KE were added in 50 μL glycine buffer (0.1M) at pH 2, 3, 4, 5, and 6 and incubated at 4° C. for 15 min. For high-pH pre-treatment, the same amount of enzyme was incubated with Tris-HCl buffer at pH 8, 10 and 12 at 4° C. for 15 min. After pretreatment, 0.8 mL of 0.5% azocasein dissolved in 50 mM potassium phosphate buffer at pH 7.5 was added to measure the remaining activity of free and immobilized keratinase.

Heat stabilities of free and immobilized KE were compared. Two μg of free KE or 100 μg of immobilized KE in 50 μl K-PO₄ buffer, pH 7.5 were incubated at 70° C., 80° C., and 90° C. for 1, 5, and 10 min. The residual enzyme activity was determined by the azocasein assay as previously described. To compare the durability of soluble and immobilized enzyme, 100 μg of free KE and 500 mg of immobilized beads were added separately in two tubes containing 10 mL of 50 mM potassium phosphate buffer pH 7.5 at 50° C. Sodium Azide (0.01%) was added to prevent microbial growth. At different time intervals, free and immobilized KE were taken for keratinase activity analysis.

### Example 15

### Protein hydrolysis.

Feather keratin, casein and bovine serum albumin (BSA) were used as substrates to examine their degradation by the immobilized keratinase. Immobilized KE (1.0 mg) was incubated with 25 mL of 1% BSA, casein or feather keratin in 50 mM K-PO₄ buffer, pH 7.5, at 50° C. under constant mixing in a 50 mL flask. Aliquots were collected and centrifuged. The aliquots were filtered and analyzed for free amino groups using the ninhydrin method previously described. The BSA and α-casein were purchased from Sigma Chemical Co (St. Louis, Mo.).

### Example 16

### Construction of expression vectors.

Plasmids, pJB and pJC, were constructed for the expression in B. subtilis. Fragment kerA, without the termination sequence and STOP codon, containing KpnI and BamHI restriction enzyme sites were amplified by PCR, using primers KERKpnI (SEQ ID NO: 3) and KERBamHI (SEQ ID NO: 4) (Table 1). This allowed in-frame fusion with the full length of stp (496 bp), in which the START codon was mutated and an aspartic acid codon was introduced as a linker. The same method was used to generate pJC harboring the 360 bp core-streptavidin gene (stpc) fused with kerA (FIG. 1). In E. coli, mature region [symbol] kerA, 840 bp, of kerA was amplified from the PWD-1 genome and cloned into expression vectors pET 26(+), pETSA7 and pETSAC10, generating pKER, pKSTP and pSTPCK, respectively. Also, the pro- and prepro-regions were inserted at N-terminal of [symbol] kerA and creating pProK, pPreProK, pProKSTP, pPreProKSTP, pProKSTPC, and pPreProKSTPC (FIG. 2).

The inserted fusion genes were analyzed and identified by three different methods, including restriction enzyme digestion, colony PCR amplification, and DNA sequencing. To assure that no nucleotide mutation was introduced during PCR amplification, all constructed plasmids were prepared and purified for DNA sequencing. The sequences of fusion genes were confirmed to be identical to those of previously reported kerA (Lin et al. (1995) Appl. Env. Microb. 61:1469-1474) and stp (Argarana et al. (1986) Mol. Biotech. 6:53-64).

### Exmaple 17

### Expression in B. subtilis.

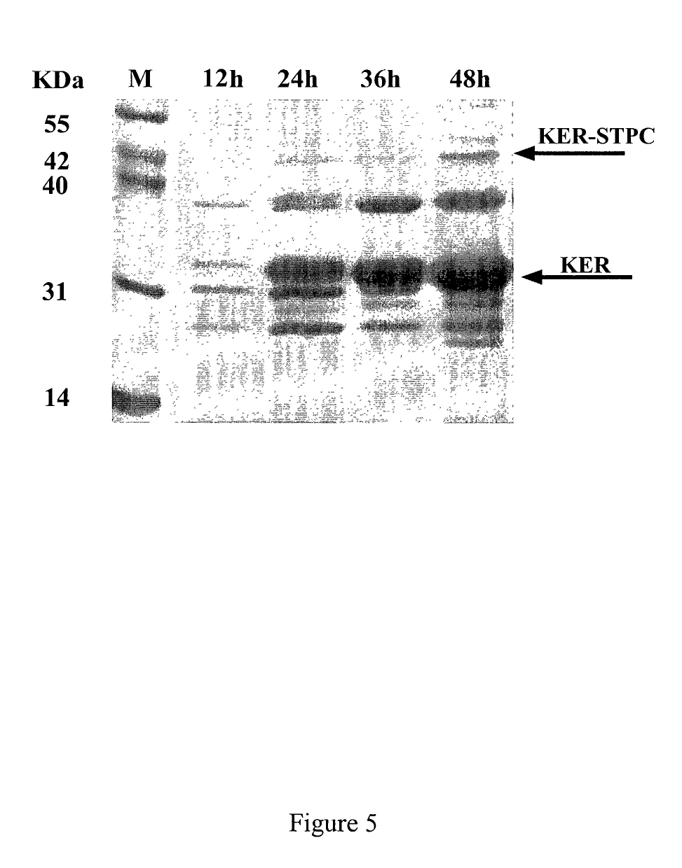

The expression of fusion proteins was examined by the measurement of keratinolytic activity and analysis by SDS-PAGE (Table 2). Keratinase activity was detected in both pJB/DB 104 and pJC/DB104 recombinants. However, fusion protein was not detectable by SDS-PAGE of pJB/DB104. Analysis of the medium supernatant collected at different culturing times of cells transformed with pJC/DB104 indicated that the fusion protein from pJC/DB104 was constitutively expressed. However, STPC (STP core protein) and the fusion protein were degraded while mature KER increased with time (FIG. 5).

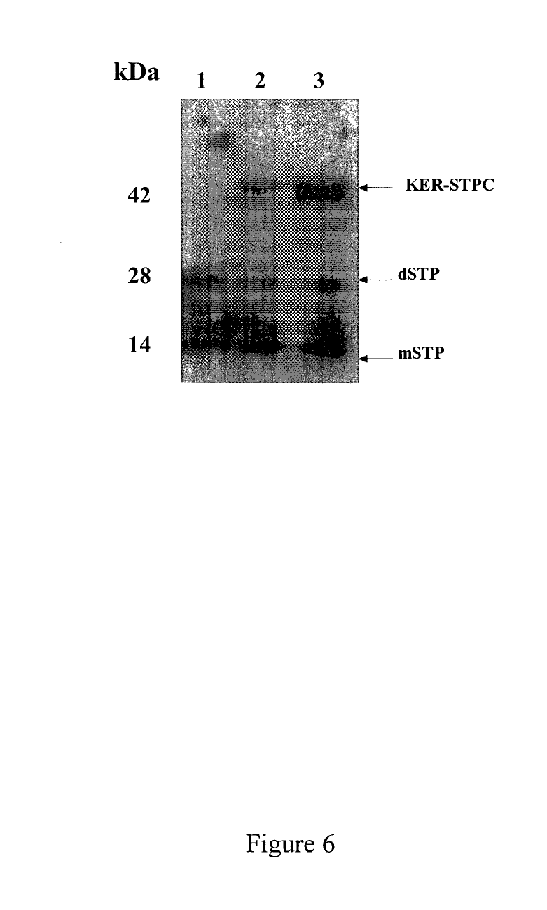

Modification of the linker sequence was used to improve the yield of fusion protein from Bacillus. The nucleotides coding for the last four amino acids (376-379 from SEQ ID NO: 2) at the C-terminal of keratinase were deleted and conjugated in-frame with stp or stpc, generating plasmids pJBD or pJCD. The fusion protein expressed from pJBD was found to be as sensitive as that from pJB. In contrast, the yield of intact fusion protein from pJCD with the four amino acid deletion increased as compared to the yield from pJC (FIG. 6). Degradation of fusion protein still occurred, though with a lesser degree.

### Example 18

### Expression in E. coli.

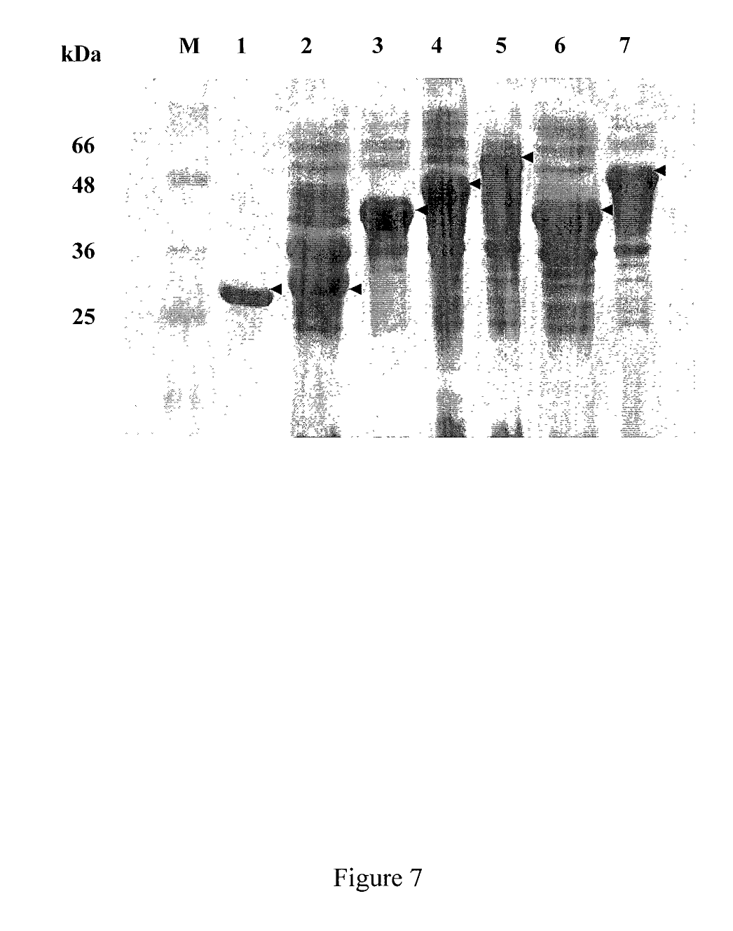

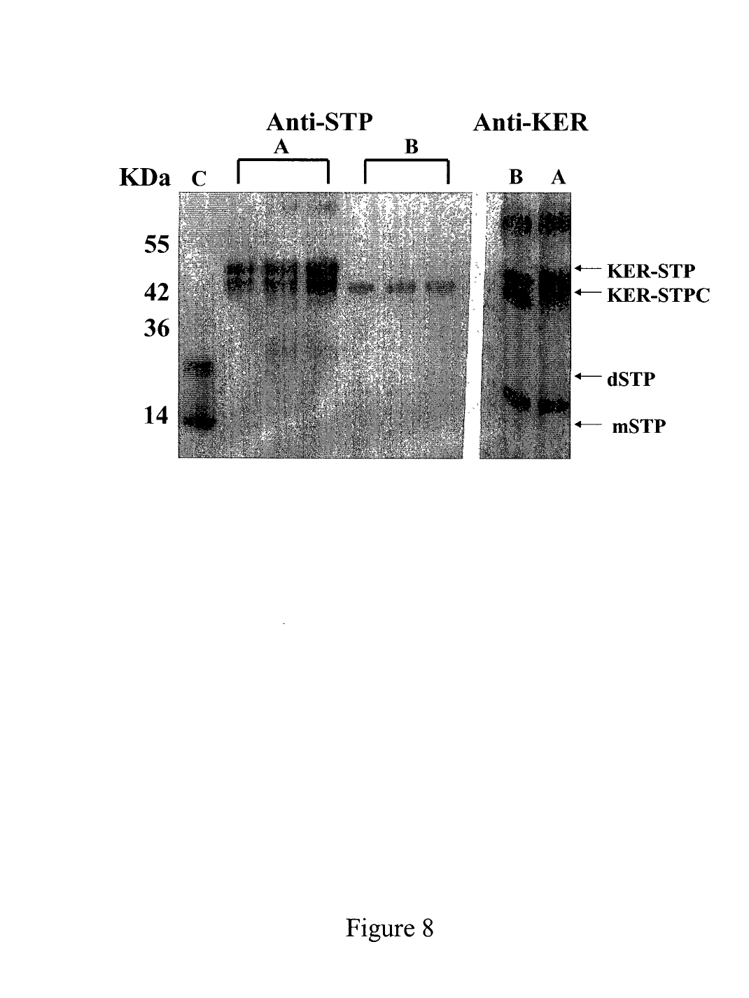

Over-expression of the intracellular KER and its fusion protein was observed under the induction of T7/lac promoter by IPTG (FIGS. 7 and 8). The over-expressed fusion protein was insoluble in the inclusion body fraction of E. coli (Table 3). After solubilization in 6N guanidine HCl, the solution was dialyzed against various refolding buffers (Table 4). In some buffers, for example, a mixture of

TABLE 3
Cellular fractionations and keratinase activity in transformed E. coli.

| | Protein | % total cellular | |
|---|---|---|---|
| Fraction | (mg) | protein | Keratinase Activity |
| Periplasm | 24 | 22 | − |
| Cytoplasm | 55 | 51 | − |
| Insoluble | 28.8 | 27 | + |
| (Inculsion Bodies) | | | |

TABLE 4
In vitro renaturation of pro-keratinase and pro-kertinase-strepavidin
with various refolding buffers.

| | Keratinase activity | |
|---|---|---|
| | | Pro- |
| Buffer | Pro-KER | KERSTP |
| 0.2 M Na₂HPO₄, pH 7.0 | ++ | ++ |
| 0.5 M (NH₄)₂SO₄, pH 7.0 | ++ | ++ |
| 0.4 M (NH₄)₂SO₄, 10 mM Na₂HPO₄, pH 7.0 | ++ | ++ |
| 0.5 M (NH₄)₂SO₄, , 10 mM Tris-HCl, 10 mM | +++ | +++ |
| Na₂HPO₄, 1 mM CaCl₂ pH 7.0 M | | |
| 20, 200 mM Tris-HCl, pH 8.0 | − | − |
| 20, 200 mM Tris-HCl, 0.8% NaCl, pH 8.0 | − | − |
| 20, 50 mM Na₂HPO₄, 0.8% NaCl, pH 7.0 | − | − |
| 0.05 mM Na₂HPO₄, 5% and 10% DMF, pH 7.0 | − | − |
| 0.2 M Na₂HPO₄, 5% and 10% DMF, pH 7.0 | ++ | ++ |

All refolding processing were performed by dialysis of 0.1 mg/mL pro-keratinase and pro-keratinase-strepavidin. 0.5 M (NH₄)₂SO₄, 10 mM Tris-HCl, 10 mM Na₂HPO₄ and 1 mM CaCl₂, pH 7.0 being the best, keratinase activity was recovered as an evidence of the renaturation of KER or KER-STP. The pro-region was required for the refolding process (Table 2, FIGS. 7-9).

### Example 19

### Immobilization of fusion proteins.

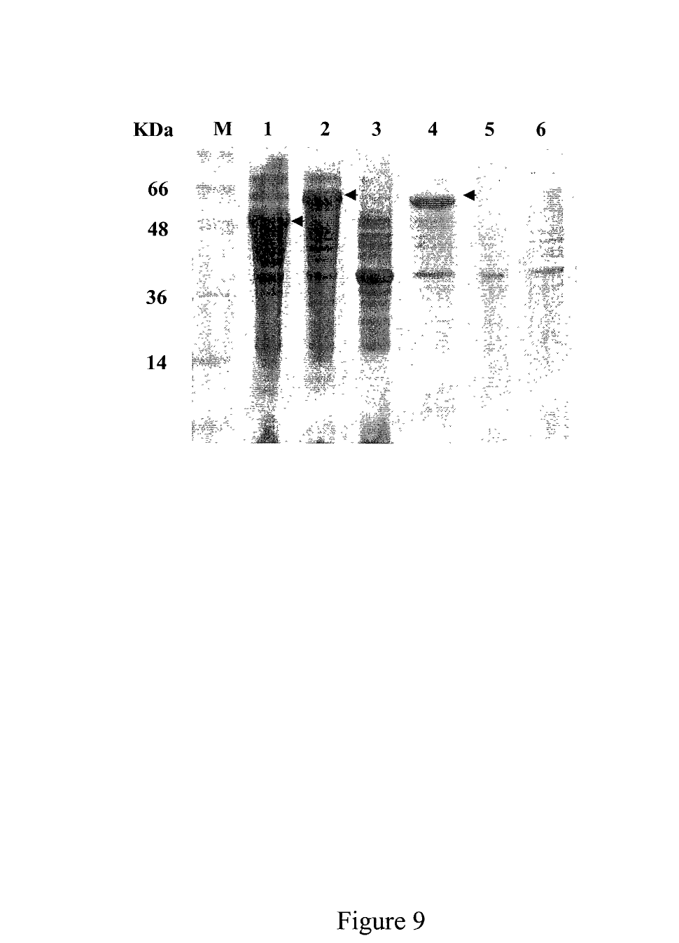

Fusion proteins produced from Bacillus and E. coli were immobilized on biotinylated matrices using different methods as described above. The binding of fusion protein from E. coli was tested by SDS-PAGE. As shown in FIG. 9, after the crude ProKER-STP fusion protein (lane 4) was mixed with biotinylated beads and dialyzed against the refolding buffer overnight at 4° C., the fusion protein disappeared from the supernatant (lane 5 and 6). Both biotinylated acrylic and agarose bound the STP-containing fusion protein equally well. Immobilized KER-STP and KER-STPC retained about 24-28% of specific keratinase activity (Table 5). B. subtilis and E. coli performed at approximately the same efficiency. However, the Bacillus system does not require the extraction and refolding process as does the E. coli system.

TABLE 5
Keratinase activity of free and immobilized
fusion proteins.

| | | Sp. Activity. | Relative Sp. |
|---|---|---|---|
| Enzyme | Host | (U/mg protein) | Act. (%) |
| Free | | | |
| Pure | B. licheniformis PWD-1 | 1600 | 100 |
| keratinase | | | |
| Pro-KER | E. coli BL21(DE3) pLysS | 1020 | 64 |
| Pro-KSTP | ″ | 760 | 47 |
| Pro-KSTPC | ″ | 860 | 54 |
| Im- | | | |
| mobilized¹ | | | |
| Pro-KSTP | E. coli BL21(DE3) pLysS | 391 | 24 |
| Pro-KSTPC | ″ | 433 | 27 |
| KSTPC | B. subtilis pJCD/WB600 | 450 | 28 |

¹Acrylic biotin beads were used. Binding capacity: 25.6 mg strepavidin/g beads.

### Example 20

### Thermal stability of immobilized keratinase.

The thermal stability of the free and immobilized keratinase was compared at three different temperatures (70, 80, and 90° C.), the results of which are shown in Table 6. The soluble enzyme was completely denatured (≦1% activity) after 5 min. incubation at all three temperatures. In contrast, the immobilized enzyme exhibited significantly greater heat stability. The immobilized enzyme retained approximately 30% activity after 10 min. at 70° C., and approximately 20% activity after 10 min. incubation at 80° C. or after 1 min. incubation at 90° C.

TABLE 6
Heat stability of soluble and immobilized keratinase in hydrolysis of
azocasein.

Relative activity, (%)

| Treatment, ° C. | Time, min | Soluble | Immobilized |
|---|---|---|---|
| Untreated | 0 | 100 | 100 |
| 70 | 1 | 53 | 86 |
| | 5 | 1 | 42 |
| | 10 | 1 | 27 |
| 80 | 1 | 24 | 35 |
| | 5 | 0 | 20 |
| | 10 | 0 | 17 |
| 90 | 1 | 2 | 21 |

### Example 21

### Enzyme activity and kinetics.

The enzyme activity and kinetic parameters of soluble keratinase, and immobilized keratinase with different substrates were determined and summarized in Table 7. Three different substrates including insoluble

TABLE 7
Specific activity of free and immobilized keratinase.

| | Azokeratin¹ | Azocasein² | Feather keratin³ |
|---|---|---|---|
| Enzyme | (Specific Activity, U/mg) | | (μmole Leu eq./mg) |
| Keratinase (KE) | 2,560 | 54,600 | 7.52 |
| Immob. keratinase | 518 | 16,412 | 1.35 |
| (IKE) | | | |
| IKE/KE | 0.20 | 0.30 | 0.18 |

¹Azokeratin assay: 1 U = an increase of A₄₅₀ with 0.01 per hour at 50° C.
²Azocasein assay: 1 U = an increase of A₄₅₀ with 0.01 per hour at 37° C.
³Ninhydrin method: increase of free amino groups as measured by leucine equivalent.

azokeratin, feather keratin, and azocasein were used for comparison. In free and soluble form, keratinase was found to have higher proteolytic activity. Immobilization reduced the keratinolytic and caseinolytic activity by 70% to 80%. Tetrapeptide AAPF was used to determine kinetic parameters (Table 8). Immobilized keratinase had decreased Vₘₐₓ, k_{cat}, and increased K_{M}. The immobilized enzyme affinity and turnover number were reduced about two- to three-fold. The overall catalytic efficiency (k_{cat}/K_{M}) was decreased about eight-fold compared with free keratinase.

TABLE 8
Kinetic parameters of the hydrolysis of tetrapeptidyl nitroanilides
(N-succinyl-AAPF-pA) by soluble and immobilized keratinase.

| | Vmax | Km | k_{cat} | k_{cat}/Km |
|---|---|---|---|---|
| Enzyme | (mM · min⁻¹) | (mM) | (min⁻¹) | (mM⁻¹ min⁻¹) |
| Soluble | 0.11 | 0.22 | 11,312 | 51,419 |
| keratinase | | | | |
| Immobilized | 0.04 | 0.76 | 5,019 | 6,604 |
| keratinase | | | | |

### Example 22

### Stability at different pHs.

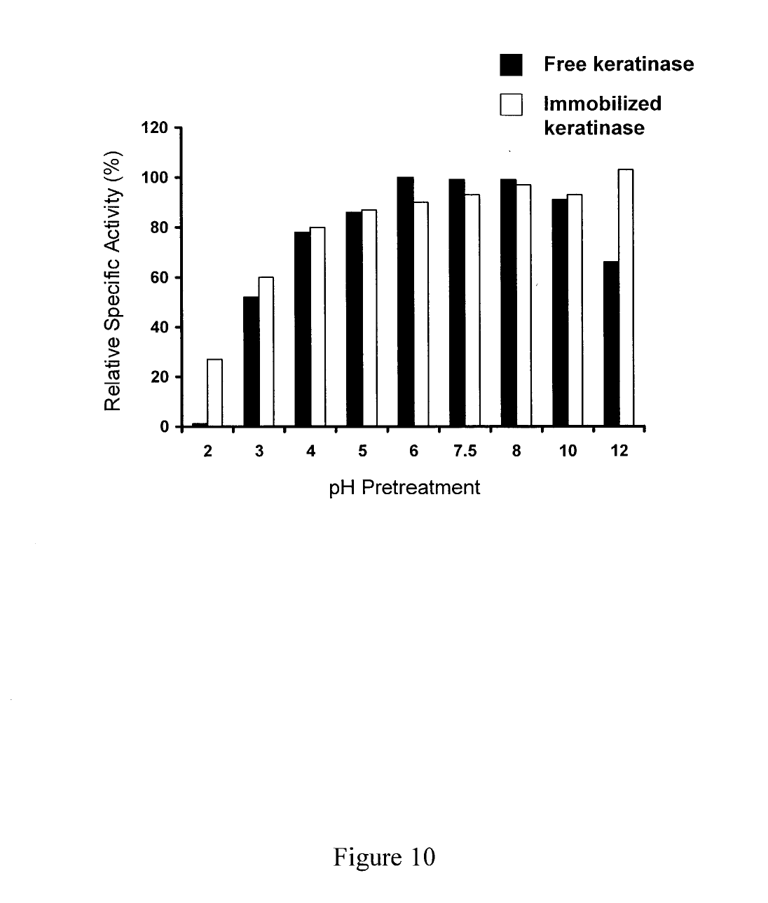

The soluble and immobilized keratinase were pretreated by buffers with low (2.0, 3.0, 4.0, 5.0, 6.0) and high (8.0, 10.0, 12.0) pH. The recovery of caseinolytic activity was compared as indicated in FIG. 10. Both free and immobilized keratinase showed sensitivity to acidic conditions but were less sensitive to alkaline pH. Compared with soluble keratinase, the immobilized keratinase was much more stable to extreme pHs. It maintained 50% enzyme activity after the treatment at pH 2.0 and 100% activity after the treatment at pH 12.

### Example 23

### pH profiles.

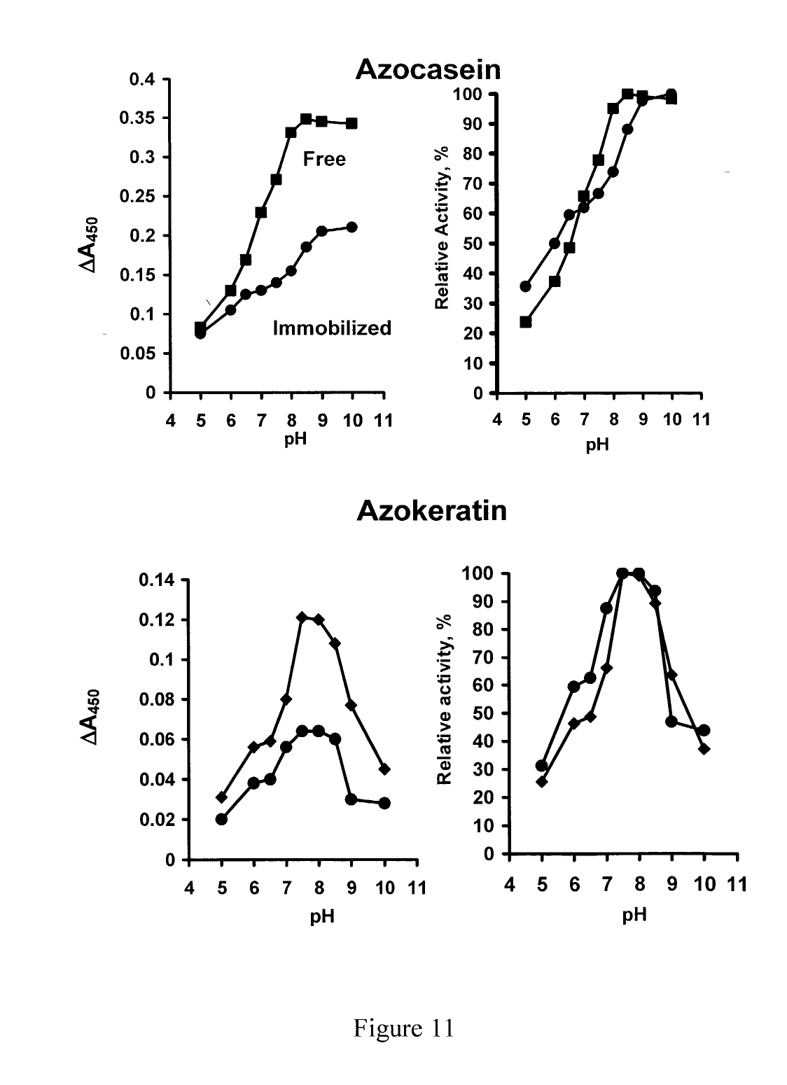

The optimal pH and pH profiles for free and immobilized keratinase with azocasein and azokeratin as substrates were shown in FIG. 11. Both free and immobilized keratinase showed different optimal pH and pH profiles with the two substrates. For azocasein, both soluble and immobilized enzyme activity increased with increasing pH, up to pH 9-10. For azokeratin, in contrast, the optimal pH was found narrowly in the neutral range, pH 7-8. Hence, the pH profile appeared to be related to the chemical nature of the substrates.

### Example 24

### Enzyme stability.

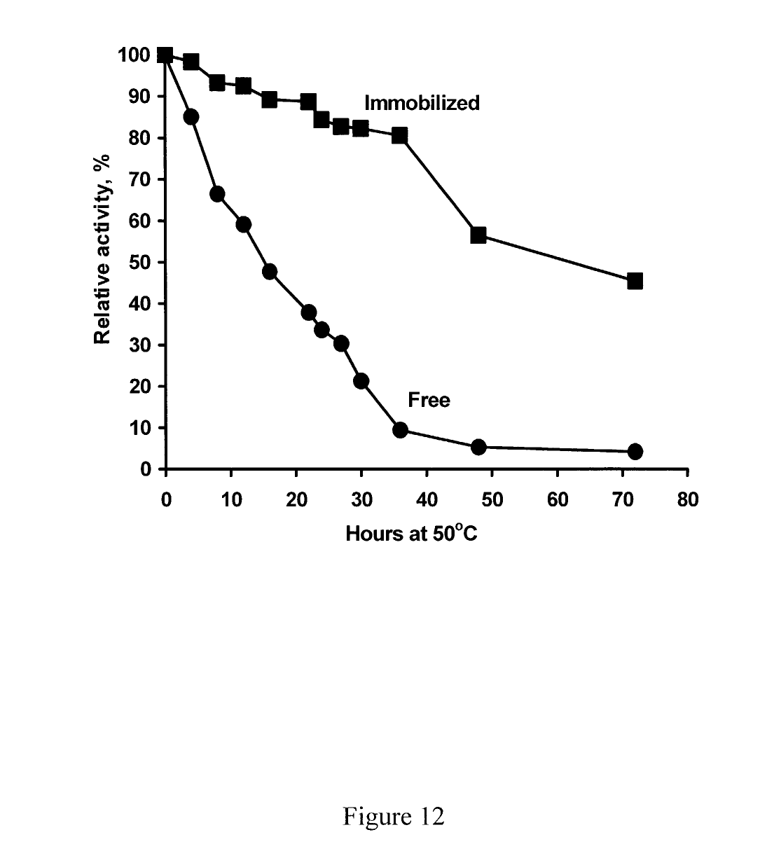

The long-term stability of KE at 50° C. (the optimal temperature) over a 3-day period was tested (FIG. 12). It was found that the enzyme stability of the immobilized KE was significantly improved. The half-life of immobilized and soluble KE was approximately 50 hr and 15 hr, respectively. After the 3-day incubation at 50° C., the immobilized KE maintained 48% activity, whereas the soluble enzyme was only 2% active.

### Example 25

### Casein and feather keratin hydrolysis.

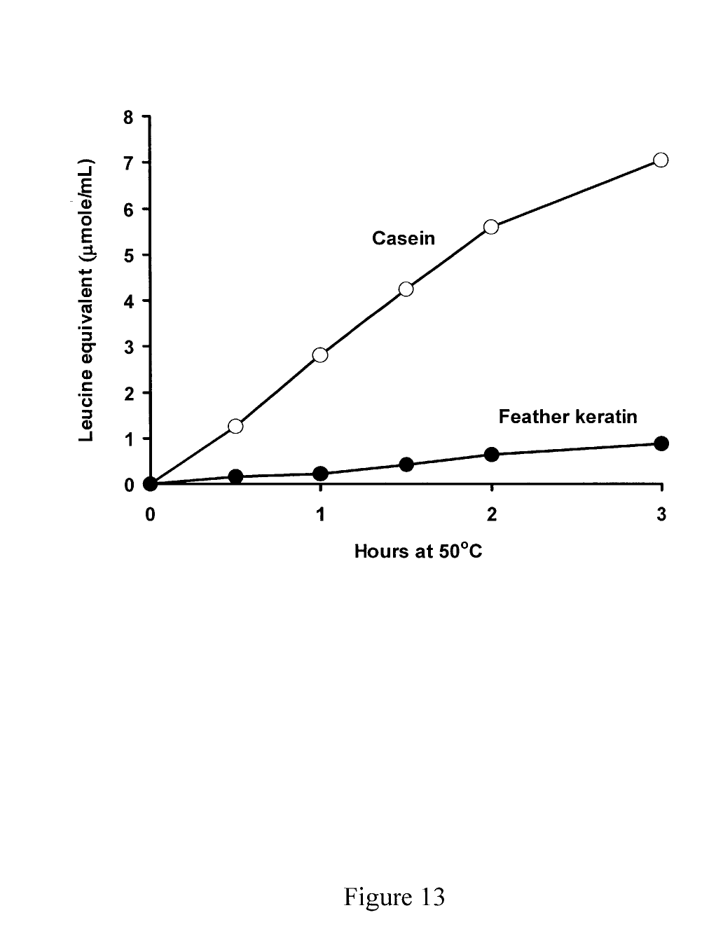

Immobilized KBR-STP was prepared and tested for the hydrolysis of casein and feather keratin (FIG. 13). Immobilized keratinase converted proteins to peptides and amino acids as indicated by the increase of free amino groups using the ninhydrin assay.

The foregoing is illustrative of the present invention, and is not to be construed as limiting thereof. The invention is defined by the following claims, with equivalents of the claims to be included therein.

That which is claimed is:

1. A recombinant nucleic acid encoding a fusion protein, said recombinant nucleic acid comprising a nucleic acid encoding a keratinase fused to a nucleic acid encoding a first member of a specific binding pair.

2. A recombinant nucleic acid according to claim 1, said nucleic acid encoding a keratinase comprising:
(a) nucleic acid encoding the Bacillus licheniformis PWD-1 keratinase;
(b) nucleic acid that hybridizes to a nucleic acid of (a) above under stringent conditions; and
(c) nucleic acid that differs from the nucleic acid of (a) and (b) above due to the degeneracy of the genetic code, and which encodes a protein encoded by the nucleic acids of (a) and (b) above.

3. A recombinant nucleic acid according to claim 2, said stringent conditions represented by a wash stringency of 0.3M NaCl, 0.03M sodium citrate, 0.1% SDS at 70° C.

4. A recombinant nucleic acid according to claim 3, wherein said nucleic acid encoding a keratinase encodes the Bacillus licheniformis PWD-1 keratinase.

5. A recombinant nucleic acid according to claim 3, wherein said nucleic acid encoding a keratinase encodes the Bacillus licheniformis NCIB 6816 subtilisin Carlsberg serine protease.

6. A recombinant nucleic acid according to claim 3, wherein said nucleic acid encoding a first member of a specific binding pair encodes avidin.

7. An expression vector comprising a nucleic acid according to claim 1 operably associated with a promoter.

8. An expression vector according to claim 7, wherein said expression vector comprises a plasmid.

9. A host cell that contains an expression vector according to claim 7 and expresses the encoded protein.

10. A host cell according to claim 9, wherein said host cell is Bacillus subtilis.

11. A host cell according to claim 9, wherein said host cell is Escheridia coli.

12. A method of making a fusion protein, comprising:
(a) providing a host cell according to claim 9, then
(b) expressing said encoded protein in said host cell; and then
(c) collecting the encoded protein.

13. A method according to claim 12, wherein said host cell is Bacillus subtilis and said encoded protein is secreted by said host cell.

14. A method step according to claim 12, wherein said collecting step is carried out by contacting said encoded protein to a solid support, said solid support having a second member of said binding pair bound thereto.

15. A method of making a fusion protein, comprising:
(a) providing a host cell according to claim 9, then
(b) expressing said encoded protein in said host cell; and then
(c) collecting the encoded protein.

16. A fusion protein encoded by a nucleic acid according to claim 1.

17. A fusion protein comprising a keratinase fused to a first member of a specific binding pair.

18. An immobilized keratinase comprising:
(a) a fusion protein according to claim 17; and
(b) a solid support, said solid support having a second member of said specific binding pair bound thereto; wherein said first member of said specific binding pair is bound to said second member of said specific binding pair.

19. An immobilized keratinase according to claim 18, wherein said solid support is a bead.

20. An immobilized keratinase according to claim 18, wherein said first member of said specific binding pair is avidin and said second member of said specific binding pair is biotin.

21. A method of digesting protein or keratin, comprising:
(a) providing an immobilized keratinase according to claim 19, and then
(b) contacting a substrate to said immobilized keratinase for a time sufficient to at least partially digest said substrate to produce a degradation product therefrom,
wherein said substrate is selected from the group consisting of protein and keratin.

22. The method according to claim 21, wherein said substrate is protein.

23. The method according to claim 22, wherein said protein is casein.

24. The method according to claim 21, wherein said substrate is keratin.

25. The method according to claim 24, wherein said keratin is feather keratin.

26. The method according to claim 23, further comprising the step of collecting said degradation product.
